(19) United States
(12) Patent Application Publication
(10) Pub. No.: US 2021/0275783 A1
(43) Pub. Date: Sep. 9, 2021

(54) BLOOD PRESSURE REGULATION SYSTEM FOR THE TREATMENT OF NEUROLOGIC INJURIES

(71) Applicants: UNIVERSITY OF UTAH RESEARCH FOUNDATION, Salt Lake City, UT (US); Wake Forest University Health Sciences, Winston-Salem, NC (US); Certus Critical Care, Inc., Sacramento, CA (US)

(72) Inventors: Michael Austin Johnson, Holladay, UT (US); Adam de Havenon, Salt Lake City, UT (US); Guillaume Hoareau, Salt Lake City, UT (US); Lucas Neff, Winston-Salem, NC (US); Timothy Williams, Winston-Salem, NC (US); Melanie McWade, Sacramento, CA (US); David Poisner, Carmichael, CA (US)

(21) Appl. No.: 17/194,053

(22) Filed: Mar. 5, 2021

Related U.S. Application Data
(60) Provisional application No. 62/986,161, filed on Mar. 6, 2020.

(51) Int. Cl. A61M 25/10

(52) U.S. Cl. CPC A61M 25/10; A61M 2025/105; A61M 2025/1052; A61M 2205/0266; A61M 2205/52; A61M 2230/30; A61M 2205/18; A61M 2205/502; A61M 2205/3344

(57) ABSTRACT

Disclosed are systems and methods for reducing blood pressure variability in a patient. The system includes an endovascular catheter having an expandable element and one or more blood pressure sensors, and a computer/processing unit configured to receive blood pressure measurements and determine a blood pressure variability metric. Upon determining that the blood pressure variability metric exceeds a given blood pressure variability threshold or falls outside a predefined range, the computer/processing unit directs a catheter controller to adjust the size of the expandable element of the catheter, thereby modulating blood pressure and reducing blood pressure variability.

### CROSS-REFERENCE TO RELATED APPLICATIONS

This application claims priority to and the benefit of U.S. Provisional Patent Application Ser. No. 62/986,161, filed Mar. 6, 2020 and titled “BLOOD PRESSURE REGULATION SYSTEM FOR THE TREATMENT OF NEUROLOGIC INJURIES”, the entirety of which is incorporated herein by this reference.

### BACKGROUND

### 1. Technical Field

The present disclosure relates generally to endovascular systems configured to regulate aortic blood flow. In particular, the disclosure relates to aortic flow regulation devices that utilize a selectively expandable element positioned within the aorta and/or medication delivery to regulate blood pressure and blood pressure variability in patients that may benefit from effective blood pressure modulation, such as patients suffering from neurologic injuries and hemodynamic collapse.

### 2. Background and Relevant Art

Approximately 795,000 people experience a stroke every year in the United States, with 140,000 deaths. Even in patients who do survive, neurologic injury is prevalent, with an annual estimated cost of $34 billion. Eighty five percent of strokes are from a clot or plaque blocking an artery and limiting blood flow to a particular region of the brain. These “acute ischemic strokes” (AIS) limit perfusion to the brain tissue and cause irreversible neuronal cell death. Pharmacologic and neuro-endovascular therapies are employed to treat this type of stroke; unfortunately, various factors often limit these therapies to only a small subset of AIS patients. These patients are treated with careful medical management, blood pressure reduction, and at times, interventions to remove the blood clot. A second type of stroke, termed “hemorrhagic stroke” occurs when a blood vessel in the brain breaks down allowing for bleeding within the brain tissues.

In stroke patients, the penumbra is the brain tissue that has reduced perfusion but is not yet irreversibly damaged; the fate of the penumbra dictates neurologic outcomes following a stroke. Therefore, research efforts have focused on identifying measures to salvage the penumbra, including optimal blood pressure goals for patients with strokes. This is a target for intervention because blood pressure in the cerebrovascular circulation often equates with perfusion to those regions. Despite multiple large multicenter randomized trials, interventions to obtain discrete blood pressure targets have repeatedly failed to improve outcomes.

There is thus an ongoing need for improved systems and methods for effectively modulating blood pressure in patient's suffering from stroke or similar neurologic injuries and thereby potentially improving outcomes of such patients.

### SUMMARY

Disclosed herein are systems and methods for modulating blood pressure variability (BVP) in a patient. In one embodiment, a blood pressure variability modulation system includes an endovascular catheter having an expandable element and one or more blood pressure sensors, and a computer/processing unit configured to cause the system to receive blood pressure measurements and determine a blood pressure variability metric. Upon determining that the blood pressure variability metric exceeds a given blood pressure variability threshold or falls outside a predefined variability range, the system adjusts the size of the expandable element of the catheter, thereby modulating blood pressure and reducing blood pressure variability.

In some embodiments, a system or method also includes a medication infusion unit configured to provide one or more blood pressure modulating medications to the patient. The system may use the received blood pressure measurements. Upon determining that the blood pressure exceeds a ceiling threshold, falls below a floor threshold, or falls outside a predefined blood pressure range, the system directs the medical infusion unit to deliver one or more medications to the patient to decrease or increase the blood pressure (e.g., mean, systolic, or diastolic blood pressure).

Medications delivered to the patient can include vasodilating medications and/or vasoconstricting medications. For example, if the blood pressure is determined to be too high (e.g., is determined to exceed a ceiling threshold or is above a predefined range), then the system can deliver a vasodilating medication, whereas if the blood pressure is determined to be too low (e.g., is determined to fall below a floor threshold or is below a predefined range), then the system can deliver a vasoconstricting medication.

In some embodiments, a system or method determines if one or more alarm conditions exist (e.g., heart rate variability that exists outside a threshold for an excessive amount of time, failure to reach the expected change in blood pressure via medication infusion and/or adjustments to the expandable element of the catheter, changes in heart rate variability that are faster than a threshold, sudden increases in intracranial pressure, or other physiological changes to the patient that call for concern or additional action), and upon determining that one or more alarm conditions exist, initiates an alarm notification at a user interface.

In some embodiments, the expandable element includes one or more balloons and/or one or more frames with attached membranes. The frame(s) may be formed from a shape-change material (e.g., nitinol). The one or more sensors included in the system may include a blood pressure sensor disposed proximal of the expandable element and/or a blood pressure sensor disposed distal of the sensor.

In some embodiments, the system includes one or more environment sensors configured to measure an environmental parameter in the vicinity of the patient. Some embodiments may include one or more patient actuators configured to interface with the patient or a patient support. A patient actuator may include an actuator for adjusting an orientation of the patient, for example.

Blood pressure variability may be calculated using various techniques. Exemplary methods involve using the standard deviation or standard error of systolic pressure, the standard deviation or standard error of diastolic pressure, the standard deviation or standard error of mean arterial pressure over a given number of heartbeats or a given time period, the coefficient of variation of systolic pressure, the coefficient of variation of diastolic pressure, the coefficient of variation of mean arterial pressure over a given number of heartbeats or a given time period, the successive variation of systolic pressure, the successive variation of diastolic pressure, the successive variation of mean arterial pressure over a given number of heartbeats or a given time period, the slope of the systolic upstroke, the slope of the blood pressure waveform from the systolic peak to dicrotic notch, the slope of the waveform from the dicrotic notch to the diastolic trough, the area of under the blood pressure curve from the end of the diastolic trough to the dicrotic notch, the area under the blood pressure curve from the peak of systole to the dicrotic notch, the area under the blood pressure waveform from the dicrotic notch to the diastolic trough, the pulse pressure as calculated as the difference from the systolic peak to the diastolic trough, or combination thereof.

In some embodiments, the system is configured to receive user input indicating one or more of a target proximal pressure, the blood pressure variability threshold, blood pressure variability calculation settings, a blood pressure ceiling threshold, or a blood pressure floor threshold. In some embodiments, the system may include an auto adjust toggle configured to enabling the user to select an automated mode or a manual mode.

Also disclosed herein are methods of using any system embodiment described herein. One exemplary method of reducing blood pressure variability in a patient suffering from a neurologic emergency, the method comprises: advancing a distal end of an endovascular catheter comprising a sensor and an expandable element to position the expandable element within an aorta of the patient suffering from the neurologic emergency; and adjusting a size of the expandable element to modulate blood pressure in areas of vasculature proximal to the expandable element and thereby reduce blood pressure variability. The neurologic emergency may involve, for example, a subarachnoid hemorrhage, a subdural hemorrhage, an epidural hemorrhage, an ischemic stroke, a hemorrhagic stroke, or diffuse axonal injury.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 illustrates an exemplary system for monitoring and modulating blood pressure variability;

FIG. 2 illustrates different components of a blood pressure waveform;

FIG. 3 illustrates an exemplary use of the system for monitoring and modulating blood pressure variability;

FIG. 4 is a flow chart of a method for monitoring and modulating blood pressure variability in a patient;

FIGS. 5 and 6 illustrate data obtained during a test of a blood pressure variability modulation system in a pig model of intracerebral hemorrhage, with FIG. 5 illustrating that automated control of an endovascular balloon positioned in the aorta reduces proximal blood pressure variability and FIG. 6 illustrating associated beneficial modulation of blood pressure volume changes; and

FIG. 7 illustrates magnetic resonance images of animals used in the study associated with FIGS. 5 and 6, showing successful induction of hematoma in the left basal ganglia and thalamus.

### DETAILED DESCRIPTION

### Introduction

Blood pressure may be controlled in real time by manipulating vascular resistance at the level of the aorta. Blood pressure as used herein may refer to the mean blood pressure, the systolic blood pressure, the diastolic blood pressure, the pulse pressure, the blood pressure as it is referenced above or below an expandable element, or combination thereof. A blood pressure measurement may include a fraction or percentage of any one or more of the described types of blood pressures. As compared to pharmaceutical interventions, this direct manipulation is a more viable method of achieving stable blood pressure. Described herein are new endovascular technologies that can achieve automated partial aortic occlusion and can be dynamically controlled in real-time to respond to a patient's physiological status. Unlike purely pharmacologic interventions, partial aortic occlusion results in mechanical augmentation of blood pressure and is nearly instantaneous.

Automated endovascular devices are capable of making rapid small changes in the resistance to blood flow within the aorta, which can then result in a rapid small change in the patient's blood pressure in the vasculature above (i.e., upstream) from the expanded element. However, the absolute amount that the blood pressure can be changed solely with endovascular devices is limited. Profound low blood pressure may not be fully corrected with even complete occlusion of the aorta. Likewise, if the expandable element of the endovascular device is fully deflated yet blood pressure continues to rise, the endovascular device cannot make further changes to decrease the blood pressure. Therefore, in some situations, the use of medications in conjunction with the endovascular devices may provide a more complete solution to maintain a steady blood pressure across a greater range of fluctuations.

Medications can be used to increase blood pressure throughout the body when the endovascular device is reaching a level of occlusion that could be detrimental to organs (distal to the point of occlusion) due to decreased blood flow and perfusions. At the other extreme, alternative medications can be used to decrease the blood pressure when blood pressure continues to be above a desired range despite minimizing the mechanical intervention from the endovascular device. While an endovascular device as described herein is capable of optimizing blood pressure through minimizing blood pressure variability over a large range of pressures, the additional use of medications can thus extend the range of blood pressure over which the patient can be treated.

Johnson et al. WO 2018/132623 (“Johnson”), which is incorporated herein by this reference, describes an automated or partially automated endovascular device that can work with medication delivery subsystems to modulate blood pressure during states of critical illness and shock, such as hemorrhaging at points distal of the expandable element. In Johnson, when detrimental physiologic states are detected, such as low blood pressure above (i.e., proximal of) the balloon or excess bleeding distal of the balloon, the balloon can inflate to increase the afterload on the heart and increase the blood pressure above the point of the balloon. The effects of the balloon catheter can also be augmented by medication delivery subsystems that can provide medications as well as intravenous fluids to increase the blood pressure of the patient.

However, systems and methods to control blood pressure variability are desirable, especially, for example, in patients suffering from neurologic emergencies (e.g., a subarachnoid hemorrhage, a subdural hemorrhage, an epidural hemorrhage, an ischemic stroke, a hemorrhagic stroke, diffuse axonal injury or traumatic brain injury). In contrast to an absolute level or target blood pressure, the blood pressure variability, or the amount that the blood pressure is changing from moment to moment, may have greater correlation to actual functional outcomes. Ensuring blood pressure homeostasis with low fluctuations in the blood pressure may be more important than a specific goal blood pressure and may help limit secondary injury to the penumbra.

However, controlling blood pressure in post-stroke patients is difficult. Many pharmacological trials have failed to achieve improved outcomes by targeting specific blood pressure goals, let alone minimizing blood pressure variability. Pharmacologic means to achieve homeostasis and reduced blood pressure variability would be more difficult as most blood pressure medications have a delayed onset of action with half-lives of minutes or even hours. Such imprecision in the onset and duration of therapy makes it impossible to control smaller, but potentially important, blood pressure fluctuations on a minute-to-minute or second-to-second basis using medications alone.

Blood pressure variability is therefore a suitable hemodynamic parameter to be analyzed and controlled for patients suffering from neurologic emergencies, such as, for example, a subarachnoid hemorrhage, a subdural hemorrhage, an epidural hemorrhage, an ischemic stroke, a hemorrhagic stroke, diffuse axonal injury or traumatic brain injury. Described herein is a system that may be effectively utilized to decrease short-term changes in a patient's physiology, such as decreasing the amount of blood pressure variability over relatively short time windows (e.g., several hours, such as about 1 hour to about 12 hours, including all values and subranges therein, to days, such as 1 day to 7 days, including all values and subranges therein). Effective reduction in patient blood pressure variability can therefore lead to improved clinical outcomes for neurologic emergency victims.

### Overview of System for Blood Pressure Variability Modulation

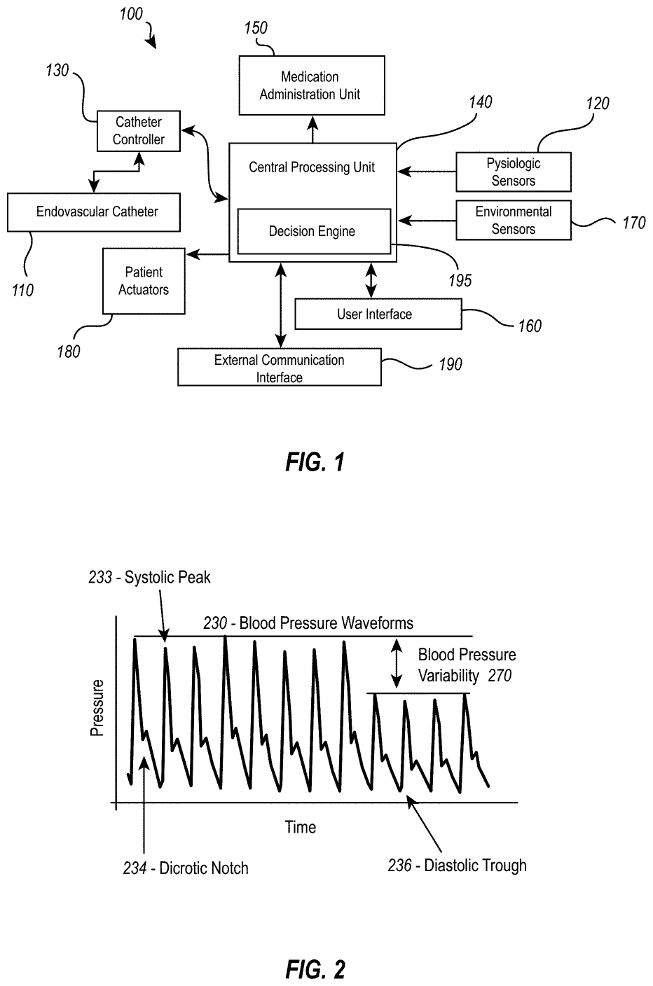

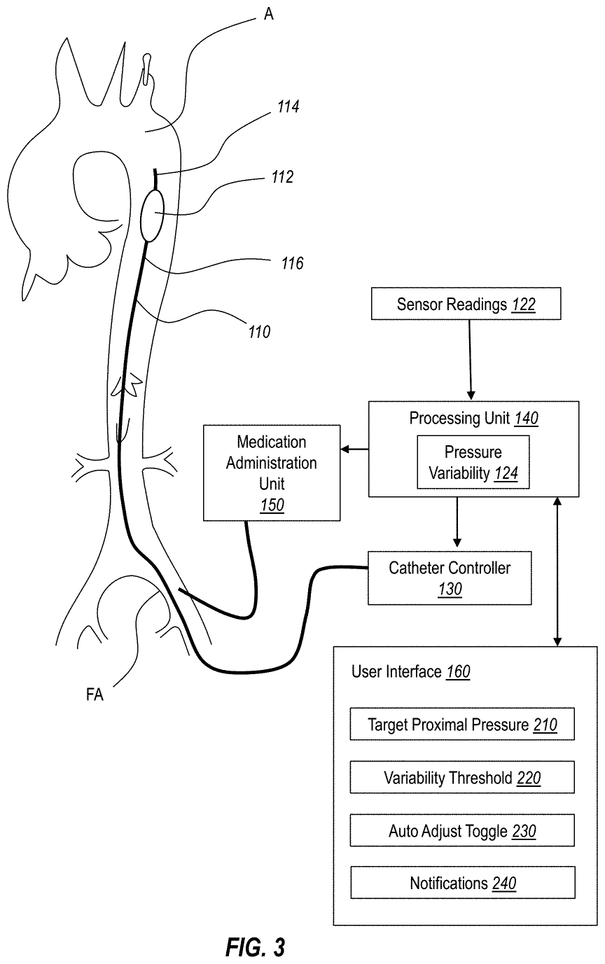

FIG. 1 schematically illustrates an exemplary system 100 for blood pressure variability modulation and management. The system 100 includes an endovascular catheter 110 operatively coupled to a catheter controller 130. The endovascular catheter 110 includes one or more expandable elements (e.g., coupled to an elongate body of the endovascular catheter), such as, for example, one or more inflatable/deflatable balloons. The catheter controller 130 functions to control aspects of the endovascular catheter 110, including, for example, controlling the size of the expandable element of the catheter to control the afterload on the heart and thereby modulate blood pressure at portions of the vasculature both distal and proximal to the expandable element (see additional description associated with FIG. 3).

For example, where the endovascular catheter 110 includes an inflatable/deflatable balloon, the catheter controller 130 may include a fluid reservoir and pump for controlling the volume of the balloon. Alternatively, a set of one or more valves may be utilized to control the flow of a biologically compatible pressurized gas, such as CO₂. In other embodiments, the expandable element may additionally or alternatively include a shape-change material (e.g., nitinol) configured to controllably expand and contract in response to applied electrical current, voltage, temperature, or pressure, for example. Such embodiments may include a frame formed from the shape-change material that is attached to one or more membranes to form a “sail” that can controllably open and close according to selective shape change of the frame. Such membranes may be made from a polymeric material suitable for contact with the aorta, for example.

The illustrated system 100 also includes one or more physiologic sensors 120. For example, one or more sensors 120 may be disposed on the endovascular catheter 110 (e.g., positioned on or integrated within an elongate body of the endovascular catheter 110) to provide physiological information related to one or more parameters. Examples of physiological sensors 120 include blood pressure sensors (proximal and/or distal to the expandable element), cranial pressure sensors, infrared signature sensors, optic nerve sensors, thermistors, blood flow sensors, tissue perfusion sensors, ultrasound transducers, emittance apparatus, and the like. One or more physiologic sensors 120 may be separate from the endovascular catheter 110. For example, one or more physiologic sensors 120 may be configured to measure heart rate, respiratory rate, blood pressure, intracranial pressure, cerebral oxygenation, cerebral blood flow, or electro-encephalographically results, and need not necessarily be coupled to the catheter 110 itself.

The central processing unit 140 includes one or more processors and memory (including working memory and one or more hardware storage devices). The processing unit 140 functions to receive input from the physiologic sensors 120, catheter controller 130, and/or other system components, and to process the various inputs to form communications and/or instructions for sending to the catheter controller 130 and/or other components of the system 100, such as a medication administration unit 150, patient actuators 180, a user interface 160, or an external communication interface 190 of an external device. Additional information related to the processing unit 140 are described below in the section titled “Additional Computer System Details.”

The system 100 may also include a medication administration unit 150 (also referred to herein as a “medication infusion subsystem”). The medication administration unit 150 is configured to administer one or more medications to the patient. The one or more medications may include one or more medications for regulating blood pressure, including vasoconstricting and/or vasodilating medications, intravenous crystalloid fluids, and/or medications to decrease ischemic or metabolic injury, for example. The medication administration unit 150 may include one or more reservoirs for each included medication, and an intravenous delivery system for intravenous delivery to the patient.

As shown, the medication administration unit 150 is also communicatively coupled to the central processing unit 140. As explained in more detail below, the processing unit 140 may be configured to cause the medication administration unit 150 to deliver one or more medications to the patient to adjust patient physiology. For example, if one or more of the physiologic sensors 120 measure a physiologic parameter outside of a predetermined range or above or below a predetermined threshold, the processing unit 140 causes the medication administration unit 150 to deliver the appropriate type and amount of medication.

For example, in some variations, the processing unit 140 causes the medication administration unit 150 to deliver medication when a measured systolic blood pressure is less than 100 mmHg (or other value within a suitable range, such as within about 85 mmHg to about 115 mmHg) and/or when a measured systolic blood pressure is greater than 180 mmHg (or other value within a suitable range, such as within about 165 mmHg to about 195 mmHg). In a particular example, when blood pressure sensors associated with the endovascular catheter 110 measure a blood pressure value that is outside of a predetermined range, above a ceiling threshold, or below a floor threshold, the processing unit 140 may cause the medication administration unit 150 to deliver a vasodilating or vasoconstricting medication to the patient.

In some embodiments the blood pressure thresholds at which the medication administration unit 150 activates may also be related to a determined state of the catheter 110. For example, a blood pressure threshold that triggers delivery of a medication, such as, for example, a vasodilating or vasoconstricting medication, may only be applicable if the processing unit 140 also determines that the expandable element of the catheter 110 is in a predefined state (e.g., expanded or collapsed) or meets a predefined state threshold (e.g., is expanded above a certain degree). In some embodiments, determining whether the expandable element meets a state threshold includes determining a gradient between the distal and proximal pressure sensors and determining if it exceeds a predetermined gradient threshold. In some embodiments, determining whether the expandable element meets a state threshold includes determining whether the level of expansion has resulted in the blood pressure below the expandable element falling below a threshold.

For example, in some variations, if the processing unit 140 determines that a gradient (i.e., difference) of distal blood pressure to proximal blood pressure is greater than or equal to about 10 mmHg to about 15 mmHg (including all values and subranges therein such as 10, 11, 12, 13, 14, or 15 mmHg, or a range with endpoints selected from any two of the foregoing values), and a measured blood pressure falls outside a predetermined range (or is above a ceiling threshold or below a floor threshold), the processing unit 140 may cause the medication administration unit 150 to deliver medication. In another variation, the processing unit 140 may cause the medication administration unit 150 to deliver medication if it determines that a measured blood pressure falls outside a predetermined range (or is above a ceiling threshold or below a floor threshold) and that further inflation of the expandable element would result in a second measured blood pressure, e.g., a blood pressure below the expandable element, to fall below a predefined threshold, such as, for example, a mean arterial pressure falling below about 65 mmHg (or other desired threshold such as within a range of about 60 mmHg to about 70 mmHg).

In yet another example, the processing unit 140 may cause the medication administration unit 150 to deliver medication if it determines that a measured blood pressure falls outside a predetermined range (or is above a ceiling threshold or below a floor threshold) and that the expandable element would be collapsed below a minimum threshold such that that further deflation of the expandable element would not have an effect on reducing the blood pressure gradient between the distal and proximal blood pressure sensors (for example if the gradient from the proximal to distal sensor is already below a threshold or is at or about 0). That is, the processing unit 140 may be configured to only deliver a particular medication if both a measured physiologic parameter (e.g., blood pressure) is above or below a physiological threshold and a particular catheter state (e.g., expanded or collapsed position of the expandable element) is detected.

More specifically, for example, if the expandable element is completely collapsed such that the distal to proximal blood pressure gradient is essentially 0 mmHg, and the systemic blood pressure is above the desired patient blood pressure setpoint (for instance the patient's blood pressure is above a systolic blood pressure of about 180 mmHg, or above the blood pressure that has been set by the user as the target pressure), vasodilatory agents (such as nicardipine, carvedilol, esmolol, or other medications that can lower a patient's blood pressure) may be introduced to effect lower systemic blood pressure to or below the desired systemic blood pressure setpoint. In some implementations, this can then allow the expandable element to activate and expand to re-establish the distal to proximal blood pressure gradient above 0 mmHg but below the predetermined gradient threshold.

Conversely, when the expandable element is expanded such that the predetermined distal to proximal pressure gradient threshold is exceeded or that the blood pressure below the expandable element is below the predefined threshold (such as a mean arterial pressure of less than about 65 mmHg or other desired threshold) and the patient's systemic blood pressure is below the desired patient blood pressure setpoint, then medications intended to raise the systemic blood pressure (i.e. norepinephrine or epinephrine, or dobutamine, or vasopressin or other vasopressor medications) may be delivered. Medications may be delivered based on either communication from the system to a healthcare provider for manual medication delivery (e.g., the system may provide an alert or instruction to a healthcare provider to manually administer medication, via, e.g., the user interface 160), or automatically via operation of the processing unit 140 and the medication administration unit 150.

The central processing unit 140 may also be communicatively coupled to a user interface 160. The user interface 160 may include a visual display, such as an LCD or LED display, audio components for audio input (e.g., microphone) and/or output (e.g., speaker(s)), other output devices known in the art, and other input devices known in the art (e.g., touch screen, buttons, mouse controller, keyboard, etc.). The central processing unit can receive and store data (e.g., measurements from any of the sensors in the system, calculations or determinations based on those measurements, user or healthcare provider profiles, system settings, and the like). For example, measurements may include but are not limited to: measured blood pressures (e.g., blood pressure measured above and below the expandable element). Calculations may include but are not limited to: blood pressure variability metrics, pressure calculations and/or predictions (e.g., the amount the blood pressure below the balloon is changing or will change in response to changes in the balloon volumes). Profiles may include but are not limited to: categorization of the severity of the blood pressure variability of the patient (e.g., high/medium/low, or as quintiles, quartiles, tertiles for a given patient population). Settings may include but are not limited to: the target blood pressure above the expandable element, the range of acceptable blood pressures above the expandable element, the minimum acceptable blood pressure below the expandable element, and/or the desired duration of the therapy. This data can be transmitted to the user via the user interface 160, through wired and/or wireless data transfer to external devices (e.g., external unit interfaces such as monitors, computers, tablets, mobile devices (e.g., smartphones), or the like.

As mentioned above, the user interface may also comprise input devices. The input devices may allow the user to provide information to the central processing unit 140 (e.g., manually). For example, a user may provide and/or select, via the input device of the user interface, target values and/or set points (e.g., one or more target blood pressures, such as, the target proximal blood pressure, the minimum distal blood pressure, etc.). A user may also provide and/or select, via the input device, limits to physiologic variables (e.g., a ceiling threshold, floor threshold, or range of blood pressures) tied to when the system should perform an action (e.g., change a size of the expandable member, deliver a medication), instruct a user to perform an action, (e.g., manually change a size of the expandable element, manually deliver medication), or take no action. Additional examples of limits to physiologic variables include but are not limited to the maximum proximal to distal pressure gradient and alarm set points, such as, maximum and/or minimum blood pressure alarms.

The system 100 may also include one or more environment sensors 170 configured to measure environmental parameters. For example, the environment sensors 170 may include an ambient barometric pressure sensor, ambient temperature sensor, a position sensor (e.g., to detect angle or tilt of patient and/or of patient support), and/or an accelerometer to determine the rate of change of the tilt of the patient support. The environment sensors 170 are communicatively coupled to the central processor 140. Measurements made by these sensors may be utilized to calibrate and/or adjust the other sensors of the system 100, and/or to determine whether to adjust the position of the patient using the patient actuators 180, for example (e.g., whether to adjust the angle of the patient's bed to decrease blood pressure to the patient's head).

The system may also include one or more patient actuators 180 configured to interface with the patient or with a patient support, such as a patient bed, gurney, stretcher or the like. In some variations, the patient actuators 180 may include actuators that control the orientation of the patient (e.g., raise or lower the patient's head in relation to the patient's trunk and/or legs, raise or lower one or more of the patient's extremities), such as, for example, mechanical arms, ratchets, pneumatic or hydraulic lifts, levers, and/or motors, or the like. Additionally, or alternatively, patient actuators 180 may modify or maintain a temperature of the patient (e.g., warm or cool a portion of the patient). Examples of these patient actuators 180 include but are not limited cooling devices such as fans, air conditioners, cooling blankets, etc., and heating devices, such as heaters, heating blankets, etcetera. In some variations, patient actuators 180 may also include audio (e.g., speakers, radios, etc.) and/or visual devices (e.g., televisions or monitors such as computer monitors, tablets, mobile devices, etc.) that provide patterns of light or displays directed at the patient's eyes, or audio output directed to the patient's ears, to help calm the patient. In some variations, one or more of the patient actuators 180 may be incorporated into the patient support (e.g., patient bed, gurney, stretcher, or the like).

In some variations, a plurality of patient actuators 180 may be used in combination and any combination of patient actuators 180 described herein may be use simultaneously or during different times throughout a treatment. For example, in one variation, systems may comprise patient actuators 180 that control the orientation of the patient and patient actuators 180 that modify or maintain a temperature of the patient. In another variation, a system may comprise a plurality of patient actuators 180 that modify or maintain a temperature of the patient (e.g., a plurality of cooling and/or heating devices positioned on different portions or regions of the patient).

The system 100 may also include an external communications interface 190 configured to allow the data (measurements, calculations, profiles, settings, and the like) of the processing unit 140 to be exported to other connected computer devices or systems (near and/or remotely connected). The interface may include a wired or wireless interface. Suitable wired interfaces include 802.3 (Ethernet), RS232, RS845, USB, HDMI, DVI, VGA, fiber optics, DisplayPort, Lightning connectors, and the like. Suitable wireless interfaces include 802.11, ultra-high frequency radio wave (e.g., Bluetooth®), and the like. The communications interface 190 may be configured to connect to a network such as a cellular network, Local Area Network (“LAN”), Wide Area Network (“WAN”), or the Internet, for example. Additional computer system connection types and network details are described below (see section titled “Additional Computer System Details”).

The illustrated central processing unit 140 also includes a decision engine 195 (i.e., system controller). The decision engine 195 functions to receive and integrate measurements from one or more of the various sensors of the system 100 (e.g., the physiologic sensors 120 and environment sensors 170), perform calculations (for instance determining the blood pressure differential between the real-time measured blood pressure and a target blood pressure, determining a distal to proximal blood pressure gradient, and/or determining the overall systemic blood pressure distal to the expandable element), and then derive various physiologic determinations based on those measurements and/or calculations (e.g., determine mean blood pressure and blood pressure variability metric and compare those values to threshold values or ranges). The decision engine 195 may further instruct and/or communicate with the appropriate actuatable components of the system based on the physiologic determinations. The resulting actions may include, for example, the catheter controller 130 adjusting the size of the expandable element of the catheter 110, the medication infusion subsystem 150 delivering one or more medications, and/or the patient actuators 180 adjusting the position or temperature of the patient).

The central processing unit 140 may also include an alarm subsystem that works in conjunction with the decision engine 195. The alarm subsystem functions to receive measurements from one or more of the various sensors of the system 100, and to compare these values to one or more alarm conditions. If an alarm condition is determined to exist, the processing unit 140 can operate to send an alarm notification to the user (e.g., via the user interface 160). An alarm condition may include, for example, heart rate variability that exists outside a threshold for an excessive amount of time, failure to reach the expected change in blood pressure via medication infusion and/or adjustments to the catheter 110, changes in heart rate variability that are faster than a threshold, sudden increases in intracranial pressure, or other physiological changes to the patient that call for concern or additional action.

The various subsystems shown in FIG. 1 may be housed in separate structures, or may be integrated into a single chassis. That is, the actual structural relationship between the various subcomponents may vary so long as each is able to operate according to its intended function. Further, the processing modules and components of the central processing unit 140 may be combined in a single computer device or divided among multiple computer devices. Some of the processing may even be done remotely and delivered via a network or other connection to the communication interface 190.

### Exemplary Use of the Blood Pressure Variability Modulation System

FIG. 2 illustrates a series of blood pressure waveforms 230, showing standard systolic peaks 233, dicrotic notches 234, and diastolic troughs 236. These blood pressure waveforms are typical of the data that can be derived from blood pressure sensors, such as the blood pressure sensors positioned on opposite sides of the expandable element in the endovascular catheter described herein. This blood pressure and all the various components of the pressure waveform may be sensed and analyzed. Blood pressure variability 270 can occur as a result of changes in blood pressure. A blood pressure variability metric may be calculated at least in part using one or more of: the standard deviation (or standard error) of systolic pressure, the standard deviation (or standard error) of diastolic pressure, the standard deviation (or standard error) of mean arterial pressure over a given number of heartbeats or a given time period, the coefficient of variation of systolic pressure, the coefficient of variation of diastolic pressure, the coefficient of variation of mean arterial pressure over a given number of heartbeats or a given time period, the successive variation of systolic pressure, the successive variation of diastolic pressure, the successive variation of mean arterial pressure over a given number of heartbeats or a given time period.

The variability in the blood pressure may also be sensed while the system is active by looking at physiologic changes and device changes that are occurring to allow for the minimization of the blood pressure variability. For example, when the device is active, the blood pressure variability metric may include the standard deviation or standard of the mean or coefficient of variation or other measure of variability of the systolic or diastolic or mean blood pressure below the expandable element. Additionally, or alternatively, the variability may include a metric of the volume changes inside the expandable element, the changes in the pressure within the expandable element, and/or other suitable measurements of blood pressure variability according to acceptable statistical measures of deviation and/or variability.

The blood pressure variability metric may additionally or alternatively be calculated using other features of blood pressure waveform measurements, including one or more of the slope of the systolic upstroke, the slope of the blood pressure waveform from the systolic peak 233 to dicrotic notch 234, the area of under the blood pressure curve from the end of the diastolic trough 236 to the dicrotic notch 234, the area under the blood pressure curve from the peak of systole 233 to the dicrotic notch 234, the area under the blood pressure waveform from the dicrotic notch to the diastolic trough, the slope of waveform from the dicrotic notch to the diastolic trough, or the pulse pressure as calculated as the difference from the systolic peak to the diastolic trough, for example.

In some embodiments, a blood pressure variability metric may be computed using a fixed combination of two or more of the foregoing measurements. When multiple measurements are utilized in combination each measurement/metric may be separately weighted. In some embodiments, the blood pressure variability metric may be computed using time-based combinations, where some metrics are used during specific time windows or when the pressures (e.g., mean arterial pressure) are above or below some threshold, and other metrics used during other time windows or when the pressures are on the other side of some threshold. Different methods of calculating a blood pressure variability metric may be selected and/or controlled through the user interface 160.

FIG. 3 illustrates a more detailed view of the exemplary use of the system 100 described in FIG. 1. As shown, the endovascular catheter 110 is inserted into the aorta (A) via a suitable endovascular route. This will typically be done through the femoral artery (FA), though other suitable routes, such as radial access, may also be utilized. The catheter 110 is inserted until the expandable element 112 is positioned at a desired location within the aorta (A), which can include Zone 1 of the aorta, Zone 2 of the aorta, or Zone 3 of the aorta. Alternatively, the device can be inserted into the iliac arteries and not advanced into the aorta.

In the illustrated embodiment, the catheter 110 includes physiologic sensors 120 in the form of a proximal pressure sensor 114 and a distal pressure sensor 116 disposed on opposite sides of the expandable element 112. Other sensors can include pressure sensors to measure the pressure inside the expandable element 112. Other embodiments may include additional sensors and/or other types of sensors, such as additional pressure sensors at other locations along the catheter 110, and/or any of the other types of physiologic sensors described above.

Note that the terms “proximal” and “distal,” as used herein in relation to sensors and/or particular localized blood pressure readings, refer to blood flow directionality from the heart. That is, “proximal” is closer to the heart while “distal” is further from the heart. This is not to be confused with the reversed usage of the terms when described from the perspective of a medical device such as a catheter, where the “distal end” of the medical device would commonly be understood as the end with the expandable element 112 furthest from the catheter controller 130 and the “proximal end” would be understood as the end closer to the operator.

The user may utilize the user interface 160 to select inputs such as a target proximal pressure 210, a blood pressure variability threshold 220, and an auto adjust toggle 230. The auto adjust toggle 230 may consist of a switch or other input device that allows a user to select an automated mode or a manual mode and indicates to the system whether it should work in an automated mode or a manual mode. In the automated mode, the central processing unit may automatically control and adjust the size of the expandable element, while in the manual mode, changes to the size of the expandable element are controlled by the user directly. In the manual mode, the processing unit may generate and provide instructions and/or recommendations to a user as to how to adjust the expandable element. These instructions and/or recommendations may be displayed to a user via, e.g., the user interface. Other inputs and selectable options may also be included, such as particular physiological alarm limits, blood pressure variability metrics and/or calculation settings, and blood pressure ceiling and floor thresholds for determining when delivery of medicine is appropriate, as described more herein. Other processing unit 140 settings described herein may also be configured using the user interface 160.

Blood pressure sensor readings 122 from the sensors 114 and 116 are sent to the processing unit 140, which determines a pressure variability metric 124 and determines whether blood pressure needs to be momentarily increased or decreased to reduce blood pressure variability. Corresponding instructions are sent to the catheter controller 130, which operates to adjust the expandable element 112, such as by adding or removing gas, liquid, or other fluid medium to or from a balloon structure, by causing a change of shape to a wire frame of the expandable element 112, or otherwise controlling the size/volume of the expandable element 112.

If the processing unit 140 determines that the blood pressure variability metric 124 is greater than the variability threshold 220, and the auto adjust toggle 230 is set to automatic, the processing unit 140 will instruct the catheter controller 130 to adjust the expandable element 112 accordingly with the intent of bringing the blood pressure variability metric 124 below the variability threshold 220. For example, in one variation in which the blood pressure variability metric is systolic blood pressure standard deviation, the variability threshold may be 10 mmHg. In this variation, if the patient's real-time systolic blood pressure standard deviation is 20 mmHg and the auto adjust toggle 230 is set to automatic, the processing unit 140 may instruct the catheter controller 130 to adjust the expandable element 112 to decrease the systolic blood pressure standard deviation through increases or decreases in the size of the expandable element. If the processing unit 140 determines that the blood pressure variability metric 124 is greater than the variability threshold 220, and the auto adjust toggle 230 is not set to automatic, the processing unit 140 may provide a notification 240 to the user, via the user interface 160, recommending that the user perform a manual adjustment of the expandable element 112.

The processing unit 140 may also determine that the mean distal blood pressure and/or mean proximal blood pressure are outside a predetermined range, above a ceiling threshold, or below a floor threshold, and provide instructions to the medication administration unit 150 to deliver appropriate medication(s) for bringing blood pressure within a desired range. As with the catheter controller 130, this may be carried out automatically, or the processing unit 140 may deliver a notification 240 to the user via the user interface 160 describing the recommended action. As described above, such ceiling and/or floor blood pressure thresholds and ranges may also be tied to a device state such that they are only applicable if the expandable element 112 also meets a required state definition (e.g., expanded or collapsed to a threshold degree).

For example, if the processing unit 140 receives measurements from the sensors or otherwise determines from received measurements that a patient has a systolic blood pressure greater than 180 mmHg and that medications to lower blood pressure are needed, the processing unit 140 may instruct or otherwise cause the medication administration unit 150 to deliver such medication (e.g., nicardipine, nifedipine, carvedilol, esmolol, nitroprusside). As another example, if the processing unit 140 receives measurements or otherwise determines that a patient a patient's real-time measured systolic blood pressure (e.g., 100 mmHg) is below a target blood pressure, the processing unit 140 may instruct or otherwise cause the medication administration unit 150 to deliver medication to increase a patient's blood pressure (e.g., norepinephrine, epinephrine, vasopressin). While described in the example as utilizing systolic blood pressure, it should be appreciated that any of the physiologic metrics described herein could be utilized to trigger administration of medication, such as, for example, physiologic metrics related to diastolic blood pressure or mean arterial blood pressure.

The processing unit 140 may also be configured to make recommendations as to changes of the target proximal pressure 210. The processing unit 140 may also be configured to automatically make adjustments to the expandable element 112 or automatically adjust medication administration with the aim to reach a calculated or user-selected target proximal pressure 210. The processing unit 140 may be configured to take additional or alternative actions upon determining that one or more of a threshold blood pressure variability, blood pressure ceiling threshold, or blood pressure floor threshold metric has been passed, such as providing an alarm notice and/or adjusting patient actuators 180.

### Exemplary Method of Reducing Blood Pressure Variability

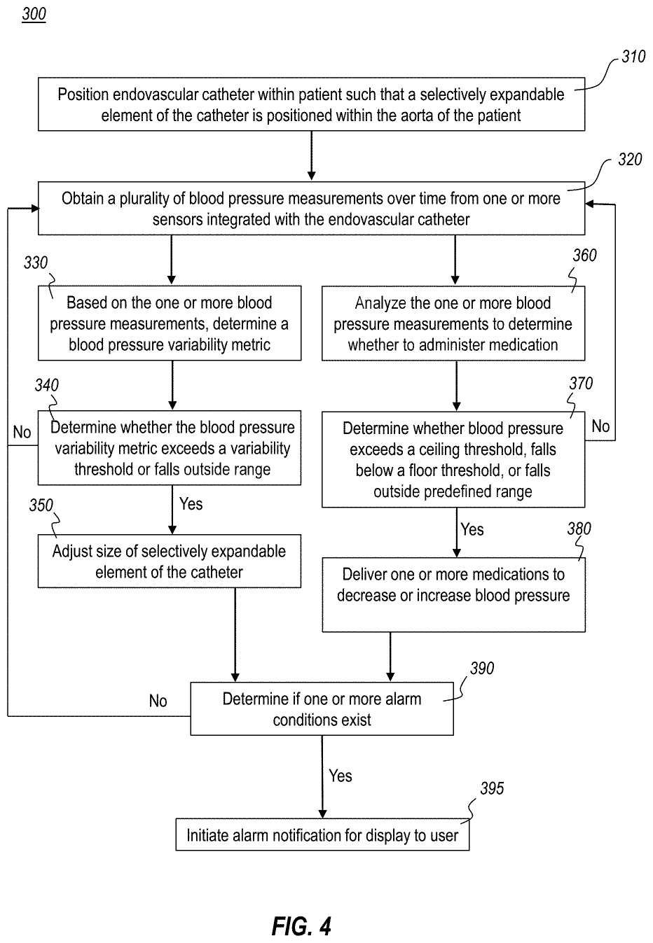

FIG. 4 illustrates a method 300 of reducing blood pressure variability. The method 300 may be carried out using the system 100 described above in relation to FIGS. 1-3, and reference numbers relating to FIGS. 1-3 are thus included in the following description as examples of corresponding structure. The method 300 may be carried out by positioning the endovascular catheter 110 within the patient such that a selectively expandable element 112 of the catheter is positioned within the aorta of the patient (step 310). The patient may be suffering from, for example, a neurologic emergency. Exemplarily neurologic emergencies may include, but are not limited to a subarachnoid hemorrhage, a subdural hemorrhage, an epidural hemorrhage, an ischemic stroke, a hemorrhagic stroke, or diffuse axonal injury. A computer system (e.g., processing unit 140) may obtain a plurality of blood pressure measurements in real-time, e.g., continuously or at predetermined intervals, during the course of treatment from one or more sensors (e.g., sensors 114, 116, 120), which may be disposed on or otherwise integrated with the endovascular catheter 110 (step 320).

Based on the one or more blood pressure measurements (e.g., systolic blood pressure, diastolic blood pressure, mean blood pressure), the computer system can adjust the size of the expandable member to reduce blood pressure variability. For example, in some variations, the computer system can determine a blood pressure variability metric (step 330), and determine whether the blood pressure variability metric exceeds a variability threshold or falls outside a predefined variability range (step 340). If the blood pressure variability metric does not exceed the variability threshold or fall outside the predefined variability range, the system may continue obtaining further blood pressure measurements. If, however, the blood pressure variability metric exceeds the variability threshold or falls outside the predefined variability range, the method may be further carried out by adjusting the size of the selectively expandable element 112 of the catheter 110 (step 350). For example, in variations in which the expandable element 112 is a balloon, adjusting the size of the expandable element may comprise inflating and/or deflating the balloon via, for example, the catheter controller 130. Thus, in these variations, the size (e.g., volume) of the balloon may be adjusted to control or modulate (e.g., decrease) the blood pressure variability.

The method may additionally include a step of analyzing the one or more blood pressure measurements for determining whether to administer one or more medications to the patient (step 360). In the illustrated method, this includes determining whether the blood pressure exceeds a ceiling threshold, falls below a floor threshold, or falls outside a predefined range (step 370). If the blood pressure does not exceed a ceiling threshold, fall below a floor threshold, or fall outside a predefined range, the method may continue obtaining further blood pressure measurements. If, however, the blood pressure does exceed a ceiling threshold, fall below a floor threshold, or fall outside a predetermined range, the method may be further carried out by delivering one or more medications to decrease or increase the blood pressure (step 380). The blood pressure to be decreased or increased can be measured as mean, systolic, or diastolic blood pressure.

Additionally, or alternatively, the method may include determining, based on blood pressure measurements and an expandable element state, whether the expandable element is or will be able to make the patient's blood pressure meet the target blood pressure through adjustment of (e.g., inflation/deflation, expansion/retraction) the expandable element. If it is determined that the patient's blood pressure can be controlled adequately by adjustment of the expandable element, the system may continue to monitor the patient's blood pressure and the expandable element state and may adjust the expandable element as needed to control the patient's blood pressure (e.g., maintain mean blood pressure within a target range, above a target floor threshold, or below a target ceiling threshold). If, however, it is determined based on the patient's blood pressure measurements and the expandable element state, that the patient's blood pressure cannot be controlled solely by adjusting the expandable element, then one or more medications to decrease or increase the patient's blood pressure (e.g., mean blood pressure) may be administered.

The method may also include the step of determining if one or more alarm conditions exist (step 390), and if so, initiating an alarm notification for communication to the user (step 395) (e.g., visually and/or audibly, via the use interface). As described above, alarm notifications may include blood pressure variability that exists outside a threshold for an excessive amount of time (e.g., for more than 10% of the total treatment time), failure to reach the expected change in blood pressure variability via medication infusion and/or adjustments to the catheter 110, changes in blood pressure variability that are faster than a threshold, or other physiological changes to the patient that call for concern or additional action.

The methods described herein may result in a reduced blood pressure variability of from about 5 mmHg to about 25 mmHg, including all values and subranges therein. For example, the methods may result in a reduced blood pressure variability of at least about 5 mmHg, about 6 mmHg, about 7 mmHg, about 8 mmHg, about 9 mmHg, about 10 mmHg, about 11 mmHg, about 12 mmHg, about 13 mmHg, about 14 mmHg, about 15 mmHg, about 16 mmHg, about 17 mmHg, about 18 mmHg, about 19 mmHg, about 20 mmHg, about 21 mmHg, about 22 mmHg, about 23 mmHg, about 24 mmHg, or about 25 mmHg or within a range with endpoints selected from any two of the foregoing values. In some variations, the methods may result in a reduced blood pressure variability of from about 5 mmHg to about 10 mmHg, from about 5 mmHg to about 15 mmHg, from about 5 mmHg to about 20 mmHg, from about 10 mmHg to about 20 mmHg, from about 10 mmHg to about 25 mmHg, from about 15 mmHg to about 20 mmHg, or from about 15 mmHg to about 25 mmHg, or within a range with endpoints selected from any two of the foregoing values. The methods described herein may additionally or alternatively result in a decrease as a percentage of the blood pressure variability metric used relative to a baseline value of the blood pressure variability metric (i.e., the blood pressure variability metric determined based on measurements before or without medical intervention/treatment). For example, in some instances, the methods described here may result in a decrease in the blood pressure variability metric by from about 25% to about 85%, including all values and subranges therein. For example, the methods described herein may result in a decrease in the blood pressure variability metric by as much 85%, 75%, 65%, 55%, 45%, 35%, 25%, or 15%, or within a range with endpoints selected from any two of the foregoing values.

### Additional Computer System Details

It will be appreciated that computer systems are increasingly taking a wide variety of forms. In this description and in the claims, the terms “controller,” “computer system,” “processing unit,” or “computing system” are defined broadly as including any device or system—or combination thereof—that includes at least one physical and tangible processor and a physical and tangible memory capable of having thereon computer-executable instructions that may be executed by a processor. By way of example, not limitation, the term “computer system” or “computing system,” as used herein is intended to include personal computers, desktop computers, laptop computers, tablets, hand-held devices (e.g., mobile telephones, PDAs, pagers), microprocessor-based or programmable consumer electronics, minicomputers, mainframe computers, multi-processor systems, network PCs, distributed computing systems, datacenters, message processors, routers, switches, and even devices that conventionally have not been considered a computing system, such as wearables (e.g., glasses).

The memory may take any form and may depend on the nature and form of the computing system. The memory can be physical system memory, which includes volatile memory, non-volatile memory, or some combination of the two. The term “memory” may also be used herein to refer to non-volatile mass storage such as physical storage media.

The computing system also has thereon multiple structures often referred to as an “executable component.” For instance, the memory of a computing system can include an executable component. The term “executable component” is the name for a structure that is well understood to one of ordinary skill in the art in the field of computing as being a structure that can be software, hardware, or a combination thereof

For instance, when implemented in software, one of ordinary skill in the art would understand that the structure of an executable component may include software objects, routines, methods, and so forth, that may be executed by one or more processors on the computing system, whether such an executable component exists in the heap of a computing system, or whether the executable component exists on computer-readable storage media. The structure of the executable component exists on a computer-readable medium in such a form that it is operable, when executed by one or more processors of the computing system, to cause the computing system to perform one or more functions, such as the functions and methods described herein. Such a structure may be computer-readable directly by a processor—as is the case if the executable component were binary. Alternatively, the structure may be structured to be interpretable and/or compiled—whether in a single stage or in multiple stages—so as to generate such binary that is directly interpretable by a processor.

The term “executable component” is also well understood by one of ordinary skill as including structures that are implemented exclusively or near-exclusively in hardware logic components, such as within a field programmable gate array (FPGA), an application specific integrated circuit (ASIC), Program-specific Standard Products (ASSPs), System-on-a-chip systems (SOCs), Complex Programmable Logic Devices (CPLDs), or any other specialized circuit. Accordingly, the term “executable component” is a term for a structure that is well understood by those of ordinary skill in the art of computing, whether implemented in software, hardware, or a combination thereof.

The terms “component,” “service,” “engine,” “module,” “control,” “generator,” or the like may also be used in this description. As used in this description and in this case, these terms—whether expressed with or without a modifying clause—are also intended to be synonymous with the term “executable component” and thus also have a structure that is well understood by those of ordinary skill in the art of computing.

While not all computing systems require a user interface, in some embodiments a computing system includes a user interface for use in communicating information from/to a user. The user interface may include output mechanisms as well as input mechanisms. The principles described herein are not limited to the precise output mechanisms or input mechanisms as such will depend on the nature of the device. However, output mechanisms might include, for instance, speakers, displays, tactile output, projections, holograms, and so forth. Examples of input mechanisms might include, for instance, microphones, touchscreens, projections, holograms, cameras, keyboards, stylus, mouse, or other pointer input, sensors of any type, and so forth.

Accordingly, embodiments described herein may comprise or utilize a special purpose or general-purpose computing system. Embodiments described herein also include physical and other computer-readable media for carrying or storing computer-executable instructions and/or data structures. Such computer-readable media can be any available media that can be accessed by a general purpose or special purpose computing system. Computer-readable media that store computer-executable instructions are physical storage media. Computer-readable media that carry computer-executable instructions are transmission media. Thus, by way of example—not limitation—embodiments disclosed or envisioned herein can comprise at least two distinctly different kinds of computer-readable media: storage media and transmission media.

Computer-readable storage media include RAM, ROM, EEPROM, solid state drives (“SSDs”), flash memory, phase-change memory (“PCM”), CD-ROM or other optical disk storage, magnetic disk storage or other magnetic storage devices, or any other physical and tangible storage medium that can be used to store desired program code in the form of computer-executable instructions or data structures and that can be accessed and executed by a general purpose or special purpose computing system to implement the disclosed functionality of the invention. For example, computer-executable instructions may be embodied on one or more computer-readable storage media to form a computer program product.

Transmission media can include a network and/or data links that can be used to carry desired program code in the form of computer-executable instructions or data structures and that can be accessed and executed by a general purpose or special purpose computing system. Combinations of the above should also be included within the scope of computer-readable media.

Further, upon reaching various computing system components, program code in the form of computer-executable instructions or data structures can be transferred automatically from transmission media to storage media (or vice versa). For example, computer-executable instructions or data structures received over a network or data link can be buffered in RAM within a network interface module (e.g., a “NIC”) and then eventually transferred to computing system RAM and/or to less volatile storage media at a computing system. Thus, it should be understood that storage media can be included in computing system components that also—or even primarily—utilize transmission media.

Those skilled in the art will further appreciate that a computing system may also contain communication channels that allow the computing system to communicate with other computing systems over, for example, a network. Accordingly, the methods described herein may be practiced in network computing environments with many types of computing systems and computing system configurations. The disclosed methods may also be practiced in distributed system environments where local and/or remote computing systems, which are linked through a network (either by hardwired data links, wireless data links, or by a combination of hardwired and wireless data links), both perform tasks. In a distributed system environment, the processing, memory, and/or storage capability may be distributed as well.

Those skilled in the art will also appreciate that the disclosed methods may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources (e.g., networks, servers, storage, applications, and services). The definition of “cloud computing” is not limited to any of the other numerous advantages that can be obtained from such a model when properly deployed.

A cloud-computing model can be composed of various characteristics, such as on-demand self-service, broad network access, resource pooling, rapid elasticity, measured service, and so forth. A cloud-computing model may also come in the form of various service models such as, for example, Software as a Service (“SaaS”), Platform as a Service (“PaaS”), and Infrastructure as a Service (“IaaS”). The cloud-computing model may also be deployed using different deployment models such as private cloud, community cloud, public cloud, hybrid cloud, and so forth.

### Example

The following example is illustrative only and should not be construed as limiting the disclosure in any way. Presented here is an example of the use of a system and method for reducing blood pressure as described herein. In the example, an endovascular catheter is used to decrease blood pressure variability in a pig model with intracerebral hemorrhage (ICH).

The goal of the study was to refine the ICH pig model to ensure animals exhibited the same high levels of blood pressure variability (BPV) seen clinically in patients with ICH and to test if systems and methods used to automate balloon inflation and deflation could successfully decrease blood pressure variability. A suitable ICH model should ideally exhibit systolic blood pressure (SBP) standard deviations (SD) of 9 to 22 mmHg as seen in the ATACH-2 study prior to intervention with the endovascular device. Three Yorkshire-cross pigs (60-90 kg) were tested. All animals were sedated, intubated and then anesthetized and maintained on anesthesia during the entirety of the study.

At baseline, all anesthetized pigs had SBPs less than 120 mmHg. To overcome this, all three animals received a low dose of norepinephrine to increase baseline SBP to 120 mmHg. Animals were instrumented to allow access to both venous and arterial structures. ICH was created in the animals in a multistep process. First, the pigs head was shaved and prepped sterility. Then an incision in the scalp was made until the bregma was exposed. A burr hole was then made 1 cm anterior and 1 cm lateral to the bregma. The burr hole was explored and bleeding was stopped using bone wax. A nick in the dura was then made and a 5.5F fogarty balloon catheter was inserted 1 cm into the brain. The fogarty balloon was then inflated with 1 mL of saline and kept inflated for 30 seconds. The balloon was then deflated. The catheter was then withdrawn 1-2 mm and blood was installed into this cavity through the wire access lumen of the fogarty catheter. Initially 2 mL of autologous blood was instilled over 1 min followed by a 1-minute pause. Then 5 mL of blood was instilled over 3 minutes.

Following the creation of the ICH, one of the three animals had a SBP>120 mmHg, and the remaining animals had SBP<120 mmHg. The first animal in the series had no interventions to specifically induce BPV, and the SBP SD was 6.9 mmHg. To reliably model clinical levels of BPV in ICH, we then incorporated simple periodic (q15 minutes) ventilator changes that resulted in highly reproducible alterations in cardiac preload with resulting changes in systemic blood pressure. Specifically, the inspiratory to expiratory (I:E) ratio was changed from 2:1 to 1:2 and the positive end expiratory pressure (PEEP) was simultaneously changed from 0 cmH2O to 5 cmH2O. It should be noted that unlike in humans, standard ventilation for large animals occurs with a PEEP of 0 cmH2O. These ventilator changes do not affect oxygenation or end-tidal CO2, but increase BPV.

The next two animals were tested using this revised methodology of ICH with ventilator induced BPV. We noted clinically relevant BPV levels were achieved with a SBP SD of 19.9 mmHg and 21.4 mmHg. In the third animal, an initial 4-hour treatment with the automated balloon catheter reduced SBP SD down to 3.3 mmHg and a subsequent one-hour wash out phase without the catheter resulted in a SBP SD of 21.4 mmHg. This resulted in a 6.7-fold decrease in SBP SD relative to the baseline BPV.

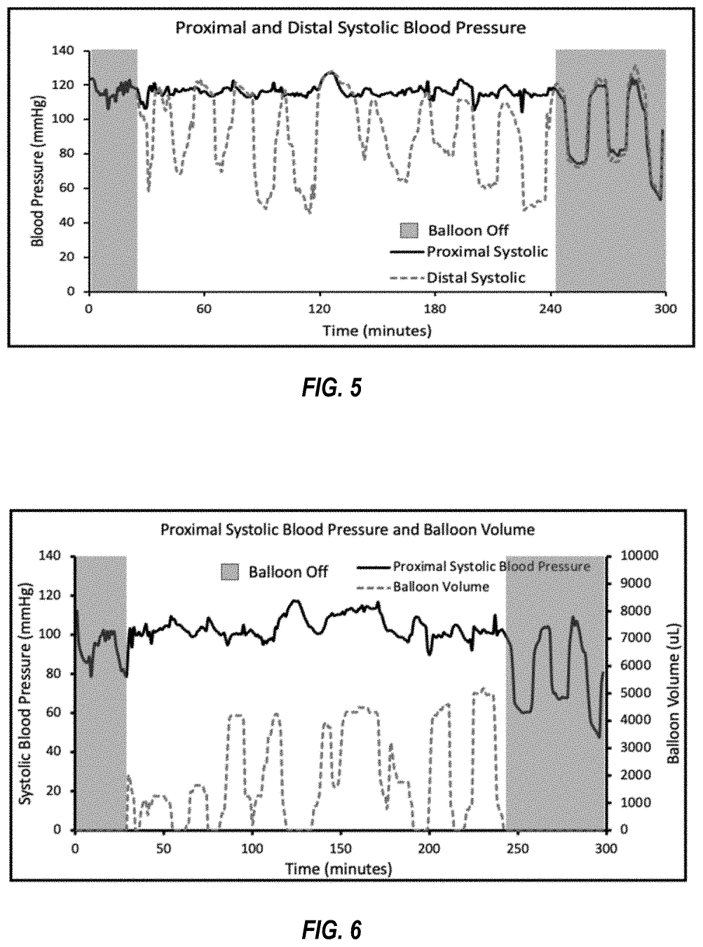

In this animal, the proximal SBP (above the balloon) during the catheter intervention portion of the study was relatively smooth and consistent (FIG. 5—Automated endovascular balloon support reduces proximal (above balloon) BPV in animal 4 from T30 to T240 minutes). In contrast, the distal blood pressure sustained repeated fluctuations as the balloon partially inflated/deflated to compensate for systemic SBP changes (FIG. 5). Since the balloon can precisely modulate to lock onto proximal pressure targets, the distal blood pressure is a helpful indicator of BPV severity without intervention. During therapy, the distal blood pressure can be analyzed in ways similar to proximal blood pressure without the intervention to calculate the amount of BPV that the device is actively controlling. Alternative methods can also be used to calculate and or estimate the inherent amount of blood pressure variability that is being controlled by an active balloon by assessing the magnitude of balloon volume changes as well as the rate of balloon volume changes that are required to minimize proximal blood pressure variability (FIG. 6—Blood pressure volume changes are an alternative metric of underlying blood pressure variability during treatment).

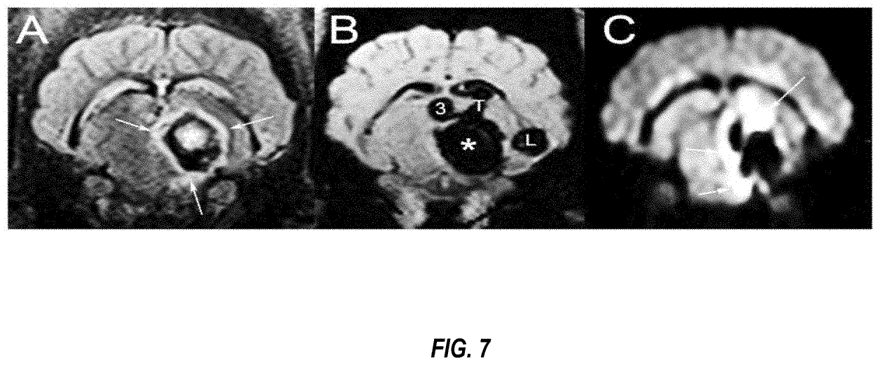

Radiographic Assessment: Magnetic Resonance images of the animals were obtained on a 3T scanner to ensure successful ICH induction (FIG. 7—coronal FLAIR (A), SWI (B), and DWI (C) MRI sequences from one of the study animals demonstrate hematoma centered in the left basal ganglia and thalamus. No ischemic injury is identified elsewhere in uninvolved structures). Hematoma volumes measured at 1.25 ml, 0.45 ml, and 0.62 ml. All animals had trace intraventricular hemorrhage and blood products along the instrument tract confirmed on susceptibility weighted imaging (SWI). Fluid attenuated inversion recovery (FLAIR) images revealed no changes in ventricular size. No infarcts were identified on DWI outside the tissue immediately adjacent to the hematoma.

Conclusion: Our pilot data indicates our ability to successfully induce ICH and clinical levels of BPV in a pig model. Furthermore, this study demonstrated our ability to reduce BPV with an automated balloon device.

### Conclusion

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, or less than 1% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may include properties, features (e.g., ingredients, components, members, elements, parts, and/or portions) described in other embodiments described herein. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

1. A system for reducing blood pressure variability in a patient, the system comprising:
an endovascular catheter comprising an expandable element configured to be positioned within an aorta of the patient and a sensor;
one or more processors; and
one or more hardware storage devices having stored thereon computer-executable instructions which are executable by the one or more processors to cause the system to at least:
obtain a plurality of blood pressure measurements from the sensor;
based on the plurality of blood pressure measurements, determine a blood pressure variability metric;
determine whether the blood pressure variability metric exceeds a variability threshold or falls outside a predefined variability range; and
upon determining that the blood pressure variability metric exceeds the variability threshold or falls outside the predefined variability range, adjust a size of the expandable element of the endovascular catheter to reduce blood pressure variability.

2. The system of claim 1, wherein the computer-executable instructions, when executed by the one or more processors, further cause the system to:
determine a blood pressure based on the plurality of blood pressure measurements;
determine whether the blood pressure exceeds a ceiling threshold, falls below a floor threshold, or falls outside a predefined blood pressure range; and
upon determining that the blood pressure exceeds the ceiling threshold, falls below the floor threshold, or falls outside the predefined blood pressure range, deliver a medication to decrease or increase blood pressure.

3. The system of claim 2, wherein the medication is a vasodilating medication or a vasoconstricting medication.

4. The system of claim 3, wherein upon determining that the blood pressure exceeds a ceiling threshold, the system delivers a vasodilating medication.

5. The system of claim 3, wherein upon determining that the blood pressure falls below a floor threshold, the system delivers a vasoconstricting medication.

6. The system of claim 1, wherein the computer-executable instructions, when executed by the one or more processors, further cause the system to determine if one or more alarm conditions exist, and upon determining that one or more alarm conditions exist, initiate an alarm notification at a user interface.

7. The system of claim 1, wherein the expandable element comprises a balloon or a frame formed from a shape-change material.

8. The system of claim 1, wherein the sensor is a proximal blood pressure sensor or a distal blood pressure sensor.

9. The system of claim 8, wherein the endovascular catheter comprises a plurality of sensors, and the plurality of sensors comprise a proximal blood pressure sensor and a distal blood pressure disposed on opposite sides of the expandable element.

10. The system of claim 1, further comprising an environment sensor configured to measure an environmental parameter in the vicinity of the patient.

11. The system of claim 1, further comprising a patient actuator configured to interface with the patient or a patient support, the patient actuator comprising an actuator for adjusting an orientation of the patient.

12. The system of claim 1, wherein the blood pressure variability metric is calculated at least in part based on the standard deviation or standard error of systolic pressure, the standard deviation or standard error of diastolic pressure, the standard deviation or standard error of mean arterial pressure over a given number of heartbeats or a given time period, the coefficient of variation of systolic pressure, the coefficient of variation of diastolic pressure, the coefficient of variation of mean arterial pressure over a given number of heartbeats or a given time period, the successive variation of systolic pressure, the successive variation of diastolic pressure, the successive variation of mean arterial pressure over a given number of heartbeats or a given time period, the slope of the systolic upstroke, the slope of the blood pressure waveform from the systolic peak to dicrotic notch, the slope of the waveform from the dicrotic notch to the diastolic trough, the area of under the blood pressure curve from the end of the diastolic trough to the dicrotic notch, the area under the blood pressure curve from the peak of systole to the dicrotic notch, the area under the blood pressure waveform from the dicrotic notch to the diastolic trough, the pulse pressure as calculated as the difference from the systolic peak to the diastolic trough, or combination thereof.

13. The system of claim 1, wherein the blood pressure variability metric is calculated at least in part based on the standard deviation or standard error of systolic pressure, the standard deviation or standard error of diastolic pressure, or the standard deviation or standard error of mean arterial pressure over a given number of heartbeats or a given time period.

14. The system of claim 1, wherein the blood pressure variability metric is calculated at least in part based on the coefficient of variation of systolic pressure, the coefficient of variation of diastolic pressure, the coefficient of variation of mean arterial pressure over a given number of heartbeats or a given time period.

15. The system of claim 1, wherein the blood pressure variability metric is calculated at least in part based on the successive variation of systolic pressure, the successive variation of diastolic pressure, or the successive variation of mean arterial pressure over a given number of heartbeats or a given time period.

16. The system of claim 1, wherein the blood pressure variability metric is calculated at least in part based on the slope of the systolic upstroke or the slope of the blood pressure waveform from the systolic peak to dicrotic notch, or the slope of the waveform from the dicrotic notch to the diastolic trough.

17. The system of claim 1, wherein the blood pressure variability metric is calculated at least in part based on the area under the blood pressure curve from the end of the diastolic trough to the dicrotic notch, the area under the blood pressure curve from the peak of systole to the dicrotic notch, the area under the blood pressure waveform from the dicrotic notch to the diastolic trough, or the pulse pressure as calculated as the difference from the systolic peak to the diastolic trough.

18. The system of claim 1, wherein the system is configured to receive user input indicating one or more of a target proximal pressure, the blood pressure variability threshold, blood pressure variability calculation settings, a blood pressure ceiling threshold, and a blood pressure floor threshold.

19. The system of claim 1, further comprising an auto adjust toggle configured to enable the user to select an automated mode or a manual mode.

20. A system for reducing blood pressure variability in a patient, the system comprising:
an endovascular catheter comprising an expandable element configured to be positioned within an aorta of the patient, a proximal blood pressure sensor disposed on a first side of the expandable element and a distal blood pressure sensor disposed on a second, opposite side of the expandable element;
one or more processors; and
one or more hardware storage devices having stored thereon computer-executable instructions which are executable by the one or more processors to cause the system to at least:
obtain a plurality of blood pressure measurements over time from the proximal and distal blood pressure sensors;
based on the plurality of blood pressure measurements, determine a blood pressure variability metric and a blood pressure;
determine whether the blood pressure variability metric exceeds a variability threshold;
upon determining that the blood pressure variability metric exceeds the variability threshold, adjust a size of the expandable element of the endovascular catheter to decrease blood pressure variability;
determine whether the blood pressure exceeds a ceiling threshold, falls below a floor threshold, or falls outside a predefined blood pressure range; and
upon determining that the blood pressure exceeds the ceiling threshold, falls below the floor threshold, or falls outside the predefined blood pressure range, deliver a medication to decrease or increase blood pressure.

21. A method of reducing blood pressure variability in a patient suffering from a neurologic emergency, the method comprising:
advancing a distal end of an endovascular catheter comprising a sensor and an expandable element to position the expandable element within an aorta of the patient suffering from the neurologic emergency; and
adjusting a size of the expandable element to reduce blood pressure variability.

22. The method of claim 21, wherein the neurologic emergency is a subarachnoid hemorrhage, a subdural hemorrhage, an epidural hemorrhage, an ischemic stroke, a hemorrhagic stroke, or diffuse axonal injury.

23. The method of claim 22, wherein blood pressure variability is reduced by at least about 5 mmHg.
